(19) United States
(12) Patent Application Publication
(10) Pub. No.: US 2026/0102269 A1
(43) Pub. Date: Apr. 16, 2026

(54) PHYSICAL THERAPY WRIST GUARD

(71) Applicant: Dongguan Xilijian Technology Co., Ltd., Dongguan City (CN)

(72) Inventor: CHANGXI HU, Dongguan City (CN)

(21) Appl. No.: 19/060,554

(22) Filed: Feb. 21, 2025

(30) Foreign Application Priority Data
Oct. 11, 2024 (CN) 202411422621.9

(51) Int. Cl. A61F 5/01

(52) U.S. Cl. CPC A61F 5/0118

(57) ABSTRACT

A physical therapy wrist guard is provided. The physical therapy wrist guard includes a wrist guard body suitable for wrapping around a hand, and providing a therapy component for treating the hand, the wrist guard body includes a base corresponding to the back of the hand, and at least one first conductive component arranged in the base, and being electrically connected to the therapy component. The control device includes a second conductive component configured for being detachably connected to the first conductive component, and an energy storage component being electrically connected to the second conductive component, after the second conductive component is connected to the first conductive component, the energy storage component supplies energy to the therapy component.

### FIELD OF THE INVENTION

The present invention relates to the field of protective clothing technology, especially to a physical therapy wrist guard.

### BACKGROUND OF THE INVENTION

Wrist guard refers to a piece of fabric used to protect the wrist joints, which has two main functions: firstly, to provide pressure and reduce swelling; secondly, to restrict activities to allow the injured area to rest and recuperate. In order to enhance the health function of wrist guards, in addition to conventional wrist guards, various types of therapy components are also installed on some wrist guards to form therapy wrist guards. During the wearing process, they can not only provide protection, but also perform therapy on the corresponding parts, greatly enhancing the health effect.

The control devices in existing therapy wrist guards are often non removable, inconvenient for charging. In addition, physical therapy wrist guards contain a large number of electronic components, which are not easy to clean. There are also some control devices in physical therapy wrist guards that can be disassembled, but have strong specificity, for example, the control device on the left wrist guard and the control device on the right wrist guard cannot be used interchangeably, making it inconvenient to use.

### SUMMARY OF THE INVENTION

### (1) Solved Technical Problem

To solve the above problems, the present invention provides a physical therapy wrist guard with a detachable control device, easy to charge, and can be connected to different wrist guard bodies; After removing the control device, the main body of the wrist guard is easy to clean and can be used as a regular wrist guard.

### (2) Technical Solution

To achieve the above objectives, the present invention provides the following technical solution:

In one aspect, a physical therapy wrist guard includes a wrist guard body suitable for wrapping around a hand, and providing a therapy component for treating the hand, the wrist guard body includes a base corresponding to the back of the hand, and at least one first conductive component arranged in the base, and being electrically connected to the therapy component. The control device includes at least one second conductive component configured for being detachably connected to the at least one first conductive component, and an energy storage component being electrically connected to the at least one second conductive component, after the at least one second conductive component is connected to the at least one first conductive component, the energy storage component supplies energy to the therapy component.

In another aspect, a physical therapy wrist guard includes a wrist guard body suitable for wrapping around a hand, and providing a therapy component for treating the hand, the wrist guard body includes a base corresponding to the back of the hand, and at least one first conductive component arranged in the base, and being electrically connected to the therapy component. The control device includes at least one second conductive component configured for being detachably connected to the at least one first conductive component, and at least one operation button to operate the wrist guard body.

### (3) Beneficial Effects

Compared with the prior art, the present invention brings the following beneficial effects:

In practical use, the wrist guard body is worn on the wrist, and the control device is connected and fixed to the base through the cooperation of the second conductive component and the first conductive component. The therapy component can be activated through the control device, so that the wrist guard body can wrap around the protective hand while also providing therapy and health care for the hand. Due to the detachable connection between the second conductive component and the first conductive component, the control device can be removed from the base to charge the energy storage component in the control device separately. In addition, as long as the wrist guard body is equipped with the first conductive component corresponding to the second conductive component, it can be adapted to the control device regardless of the type of wrist guard body, making it easy to use. Furthermore, after the control device is removed, the wrist guard body no longer has a large number of electronic components and is no longer connected to block shaped control devices, so it can be directly cleaned and worn as a regular wrist guard. Overall, by using this therapy wrist guard, the control device is detachable, easy to charge, and can be connected to different wrist guard bodies. After removing the control device, the main body of the wrist guard is easy to clean and can be used as a regular wrist guard.

Furthermore, due to the base provided at the back of the hand corresponding to the wrist guard body, the protective performance of the wrist guard body is stronger compared to ordinary wrist guards after the control device is removed.

### BRIEF DESCRIPTION OF THE DRAWINGS

FIG. 1 is a schematic structural view of a physical therapy wrist guard according to an embodiment of present invention.

FIG. 2 shows the wrist guard body of the physical therapy wrist guard of FIG. 1.

FIG. 3 is an enlarged view of the A part of the wrist guard body of FIG. 2.

FIG. 4 is an exploded view of the physical therapy wrist guard of FIG. 1.

FIG. 5 is another exploded view of the physical therapy wrist guard of FIG. 1.

FIG. 6 is an enlarged view of the B part of the physical therapy wrist guard of FIG. 5.

FIG. 7 is another schematic structural view of the physical therapy wrist guard of FIG. 1 after taking off the strap and without a therapy component according to an embodiment of present invention.

FIG. 8 shows a therapy component mounted on the physical therapy wrist guard of FIG. 7.

FIG. 9 a schematic structural view of a therapy component according to an embodiment of present invention.

FIG. 10 shows multiple first conductive components distributed in a linear array on the base.

FIG. 11 shows multiple first conductive components distributed in a square shape on the base.

FIG. 12 shows multiple first conductive components distributed in a ring shape on the base.

FIG. 13 shows the first conductive components each have a second protrusion.

FIG. 14 shows the first conductive components each have a second groove.

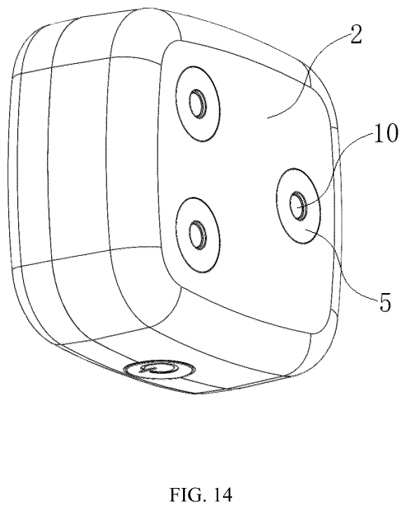

FIG. 15 shows an inner structure of the control device.

Labels in the drawing: 1, wrist guard body; 101, first finger hole; 102, second finger hole; 103, strap; 104, side opening; 105, through hole; 2, control device; 21, energy storage component; 22, shell; 23, display; 24, operation button; 25, charge port; 3, base; 4, first conductive component; 5, second conductive component; 6, first groove; 7, first protrusion; 8, chamfer; 9, second protrusion; 10, second groove; 11, therapy component; 111, connecting part; 112, therapy part.

### DETAILED DESCRIPTION OF PREFERRED EMBODIMENTS

The following will provide a clear and complete description of the technical solution in the embodiments of the present invention, in conjunction with the accompanying drawings. Obviously, the described embodiments are only a part of the embodiments of the present invention, not all of them. Based on the embodiments in the present invention, all other embodiments obtained by ordinary technicians in the art without creative labor fall within the scope of protection of the present invention.

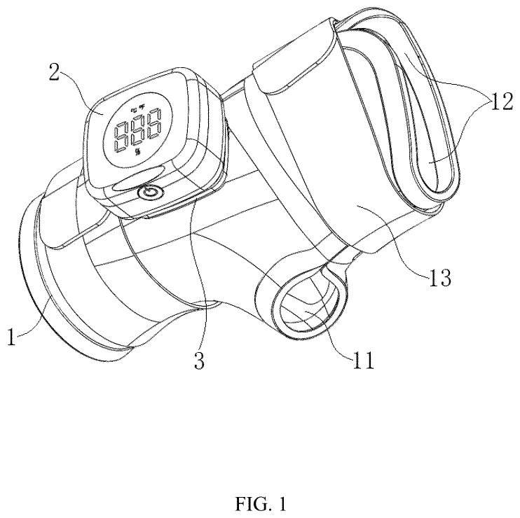

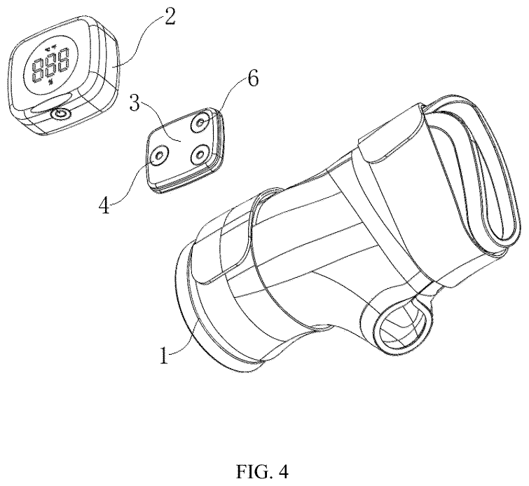

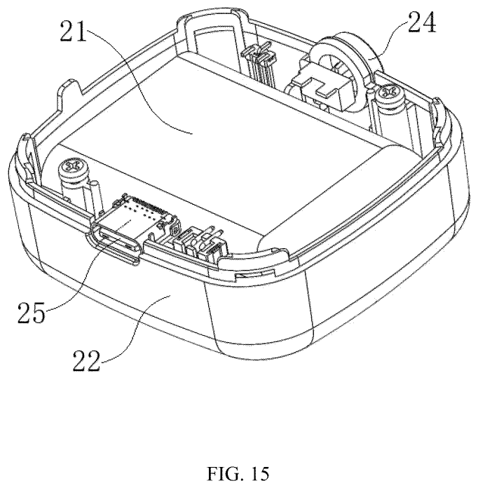

Referring to FIGS. 1-7, an embodiment of the present application discloses a therapy wrist guard, including a wrist guard body 1 and a control device 2. The wrist guard body 1 is used to wrap around the hand, and a therapy component 11 is provided on the wrist guard body 1 for performing therapy on the hand. The wrist guard body 1 is provided with a base 3 corresponding to the back of the hand, and at least one first conductive component 4 is provided on the base 3, which is electrically connected to the therapy component 11. The control device 2 is equipped with at least one second conductive component 5 corresponding to the first conductive component 4. The second conductive component 5 is detachably connected to the first conductive component 4 to fix the relative position of the control device 2 and the wrist guard body 1. In present embodiment, the control device 2 includes an energy storage member 21 (see FIG. 15), which is electrically connected to the second conductive component 5. After the second conductive component 5 is connected to the first conductive component 4, the energy storage component 21 can supply energy to the therapy component 11.

In actual use, the wrist guard body 1 is worn on the wrist, and the control device 2 is connected and fixed to the base 3 through the cooperation of the second conductive component 5 and the first conductive component 4. The therapy component 11 can be activated through the control device 2, so that the wrist guard body 1 can wrap around the protective hand and also provide therapy and health care for the hand. Due to the detachable connection between the second conductive component 5 and the first conductive component 4, the control device 2 can be removed from the base 3 to separately charge the energy storage component 21 in the control device 2. In addition, as long as the wrist guard body 1 is equipped with the first conductive component 4 corresponding to the second conductive component 5, it can be adapted to the control device 2 regardless of the type of wrist guard body 1, making it easy to use. After the removal of control device 2, since the wrist guard body 1 no longer has a large number of electronic components and is no longer connected to the block shaped control device 2, it can be directly cleaned and worn as a regular wrist guard. Overall, by using this therapy wrist guard, the control device 2 is detachable, easy to charge, and can be connected to different wrist guard bodies, and after removing the control device 2, the main body of the wrist guard is easy to clean and can be used as a regular wrist guard.

It should be noted that the shape, size, material, color and other parameters of the wrist guard body 1 are not limited in this application. They can be flexibly selected according to actual needs. As long as the wrist guard body 1 is equipped with a base 3, and the first conductive component 4 corresponding to the second conductive component 5 is provided on the base 3, regardless of the shape of the wrist guard body 1, such as left-handed wrist guard, right-handed wrist guard, finger separated wrist guard, etc., and the size, such as children's and adult models, the control device 2 can be detachably connected, so that the control device 2 can be translated and used interchangeably on different wrist guard bodies 1, making it more convenient to use.

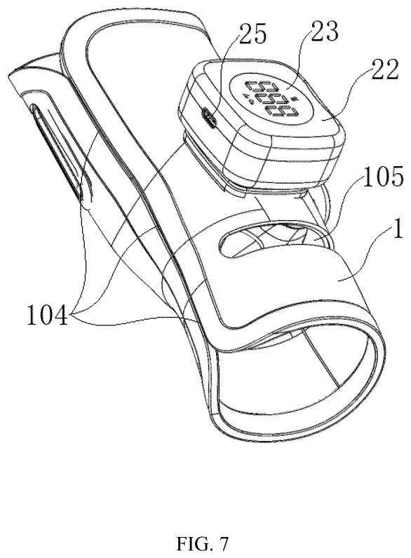

Furthermore, due to the fact that the cross-sectional size of the human hand is often larger than that of the wrist, for ordinary wrist guards with only two open ends, when inserting the hand into the wrist guard, it is necessary to first open and expand the opening at the entrance of the wrist guard, so that the hand can enter and pass through the wrist guard. This not only makes it inconvenient to wear and remove, but also requires the wrist guard to be made of deformable elastic materials, with a limited range of available materials. In order to solve the above problems, referring to FIGS. 1 and 7, the present embodiment not only has an entrance opening and an opposite exit opening of the wrist guard, but also have a side opening 104 on a side of the wrist guard body 1, making it easier for the hands to enter and withdraw the wrist guard body 1. At this time, the side opening 104 allows the wrist guard body 1 do not need to be enlarged when worn, so that the wrist guard body 1 can be made of flexible materials with elasticity or non elastic flexible materials; and the shape of the wrist guard body 1 is not limited in present application. Referring to FIG. 1, in present embodiment, the above-mentioned side opening 104 is arranged on the side of the wrist guard body 1 away from the thumb, and a first finger hole 101 corresponding to the thumb and a second finger hole 102 corresponding to the index finger, middle finger, ring finger, and little finger are formed at the exit end of the wrist guard body 1. At this time, the hand can directly enter the entrance opening of the wrist guard body 1, the thumb can be inserted into the first finger hole 101, and the remaining four fingers can directly enter the second finger hole 102. While it is convenient for the hand to insert and extract the entrance opening of the wrist guard body 1, it is also convenient for the fingers to insert and extract the exit openings, i.e., the first and second finger holes 101, 102 of the wrist guard body 1.

Based on the above scheme, referring again to FIG. 1, since the side opening 104 is set on the side of the wrist guard body 1, in order to make the wrist guard body 1 more securely worn, in present embodiment, one or two straps 13 that can wrap around the wrist and palm of the wrist guard body 1 are set up to fix the wrist guard body 1 to the hand of the human body and prevent it from falling off. The strap 13 can be fixed by magic tape, magnetic suction, snap button, button, buckle and other methods. In addition, in order to make the wrist guard body 1 adaptable and fit more sizes of hands, the material of the strap 13 is preferably a deformable elastic material.

The control device 2, in addition to the energy storage component 21, can also include components such as a shell 22, a display 23, an operation button 24, a charge port 25, etc. Furthermore, the operation button 24 can be pressed through long press, short press, single click, double click, triple click, and other methods to switch functions. Alternatively, the operation button 24 can be replaced by multiple operation buttons, or can have a keypad to have more different operations to operate the wrist guard body 1, for example to control the therapy component 11.

It should be noted that the installation position, number of components, and therapy method parameters of the therapy components 11 can be selected according to need, for example, the therapy component 11 can be set on the side of the wrist guard body 1 at a position a little away from the hand to prevent excessive therapy. Alternatively, the therapy component 11 can be placed on the side of the wrist guard body 1 near the hand, so that the therapy component 11 is close to the skin and the therapy effect is better. Alternatively, the therapy component 11 can also be placed in the interlayer of the wrist guard body 1, by this way, the therapy effect is moderate and can provide a greater degree of protection for the therapy component.

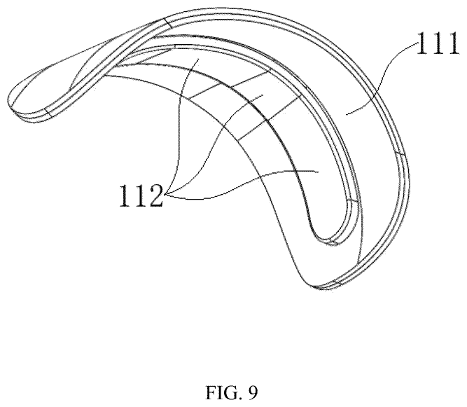

The therapy component 11 can be designed as a structure composed of a large block of therapy component 112 (see FIG. 9), which can cover a large area of the hand and perform therapy on a whole area of the hand. The therapy components 112 can also be designed as a structure consisting of several small therapy components connected in series or parallel, which can be used for targeted therapy of different parts of the hand and is more energy-efficient. Electric therapy, magnetic therapy, light therapy, ultrasound therapy, conduction heat therapy, etc. can be used in the therapy components. When using light therapy, the therapy component 112 can be arranged on the side of the wrist guard body 1 near the hand, preferably, the wrist guard body 1 can be made of transparent material to allow light to shine on the skin, or a through hole 105 (see FIG. 7) can be formed on a side of the wrist guard body 1 to allow the therapy component 112 to be more close to an therapeutic area of the hand.

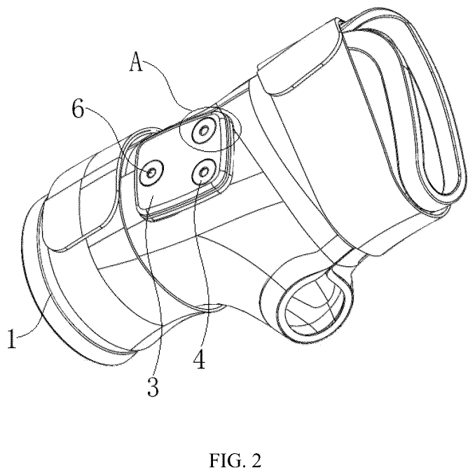

Referring to FIGS. 2-3, and 5-6, there are various ways and structures for detachable connections. One of them is introduced in this embodiment. Specifically, an extension direction of the first conductive component 4 is perpendicular to the surface of the base 3, and an extension direction of the second conductive component 5 is perpendicular to the surface of the control device 2 facing the base 3. The first conductive component 4 and the second conductive component 5 are inserted and removed to limit the movement of the control device 2 within the surface of the base 3. Both the first conductive component 4 and the second conductive component 5 are made of magnetic material and magnetically attracted to each other, which is used to limit the movement of the control device 2 in the direction perpendicular to the surface of the base 3, thereby enabling the first conductive component 4 and the second conductive component 5 to move in a direction perpendicular to the surface of the base 3. After connection, the relative position between control device 2 and base 3 is fixed.

Through the design of the above structure, after the first conductive component 4 and the second conductive component 5 are plugged and connected, the first conductive component 4 and the second conductive component 5 can only move relative to each other by moving away from each other, that is, the control device 2 can only move in the direction away from the base 3 relative to the base 3. Due to the fact that both the first conductive component 4 and the second conductive component 5 are made of magnetic material and magnetically connected, after the first conductive component 4 and the second conductive component 5 are inserted and removed, they will adhere to each other, hindering their mutual separation. This enables the first conductive component 4 and the second conductive component 5 to be firmly connected together, thereby preventing the control device 2 from detaching from the base 3 without external force.

The control device 2 has a certain weight, during the movement of the hand, the control device 2 will rotate around the elbow or shoulder with the wrist guard body 1 and the hand, and under the influence of factors such as gravity and centrifugal force, the control device 2 tends to detach from base 3, in order to solve this problem, a magnetic force between the first conductive component 4 and the second conductive component 5 is greater than a gravity of control device 2, to prevent control device 2 from detaching from base 3 without external force. Furthermore, in order to increase the magnetic force between the first conductive component 4 and the second conductive component 5, the area of the first conductive component 4 and the second conductive component 5 themselves can be increased, the number of the first conductive component 4 and the second conductive component 5 can be increased, or the magnetic grade of the material used to make the first conductive component 4 and the second conductive component 5 can be increased, so as to form a strong adhesion between the first conductive component 4 and the second conductive component 5 and prevent the control device 2 from accidentally falling off from the base 3.

Referring again to FIGS. 2-3, and 5-6, there are various ways of plug-in connection. One of them is introduced in this embodiment, specifically, the first conductive component 4 is embedded in the base 3, and the middle of the first conductive component 4 is provided with a first groove 6. The middle of the second conductive component 5 is provided with a first protrusion 7 corresponding to the first groove 6. The first conductive component 4 and the second conductive component 5 can be plug-in connected through the cooperation of the first groove 6 and the first protrusion 7.

Through the design of the above structure, after the control device 2 is removed, the first conductive component 4 is embedded in the base 3, and there are no protruding parts on the surface of the base 3, so the flatness of the base 3 can be maintained, making the base 3 usable as a regular protective plate.

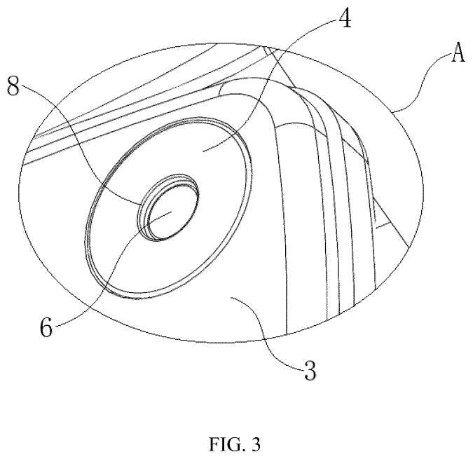

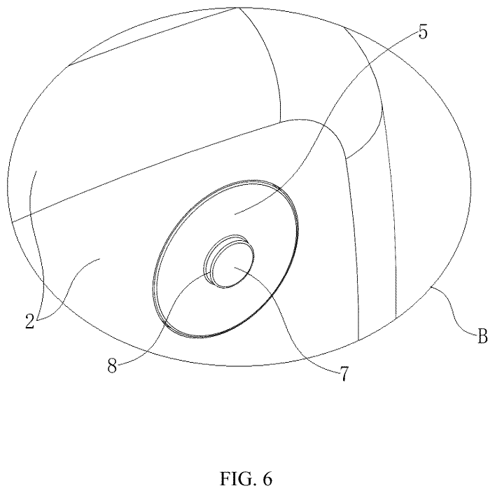

Referring to FIGS. 3 and 6, since the wrist guard body 1 needs to be adapted to the size of the hand, in order not to affect the normal movement of the hand, the thickness of the base 3 cannot be too thick, so that the depth of the first groove 6 cannot be too deep. Therefore, the thickness of the first protrusion 7 will not be too thick. Based on the above scheme, during the process of inserting the first protrusion 7 into the first groove 6, the distance between the control device 2 and the base 3 is small, which is not easy to observe directly with the naked eye. Therefore, the first protrusion 7 is not easy to insert into the first groove 6. In order to solve the above problem, the following design is carried out in this embodiment. Specifically, the side of the first groove 6 away from the wrist guard body 1 and the side of the first protrusion 7 away from the control device 2 are both provided chamfer 8 to facilitate the insertion of the first protrusion 7 into the first groove 6.

Through the design of the above structure, after setting a chamfer 8 on the side of the first groove 6 away from the wrist guard body 1, the chamfer 8 at the first groove 6 will form a frustum or frustum shape, therefore the size of the opening of the first groove 6 will significantly increase. Also, after setting a chamfer 8 on the side of the first protrusion 7 away from the control device 2, the chamfer 8 at the first protrusion 7 is similar to a cone, therefore the size of the end of the first protrusion 7 will significantly decrease. In order to complete the plug-in connection 2, the cross-sectional size of the middle of the first protrusion 7 and the middle of the first groove 6 are clearly adapted to each other, so the size of the end of the first protrusion 7 is significantly smaller than the size of the opening of the first groove 6, making it easier for the first protrusion 7 to enter the first groove 6.

Referring to FIGS. 3 and 6, it should be noted that the specific shape and quantity of the first groove 6 are not limited in this application, and can be flexibly rotated according to actual needs, for example, the cross-section of the first groove 6 is circular, and there are at least two first conductive components 4.

Through the design of the above structure, after the first protrusion 7 is inserted into the first groove 6, due to the circular cross-section of the first groove 6 and the fact that the number of first grooves 6 is the same as the number of first conductive components 4, if there is only one first conductive component 4, the first protrusion 7 and the first groove 6 will rotate relative to each other, making it impossible for the control device 2 to maintain stability relative to the base 3. Therefore, in this embodiment, when the cross-section of the first groove 6 is circular, there should be at least two first conductive components 4, at this time, according to the axiom of determining a straight line based on two points, it is possible to prevent the first protrusion 7 from rotating with each other after being inserted into the first groove 6, thereby ensuring the stability of the control device 2.

Furthermore, the cross-section of the first groove 6 can also be made non-circular. Through the design of the this structure, under the limitation of the shape of the first groove 6 itself, it is possible to prevent the first protrusion 7 and the first groove 6 from rotating relative to each other. At this time, whether the first conductive component 4 is one or multiple, it will not affect the stability of the control device 2 relative to the base 3, making it impossible for the control device 2 to rotate relative to the base 3. This ensures that during the process of inserting or removing the first protrusion 7 into or from the first groove 6, the control device 2 can only move in the direction close to or away from the base 3.

Furthermore, multiple first conductive components 4 can be set up, so that the second conductive component 5 can be connected to different first conductive components 4, thereby changing the installation direction of the control device 2. Through the design of the this structure, the direction of control device 2 can be flexibly changed, thereby changing the position of the buttons, for example the operation button 24 on control device 2 relative to the hand, the angle of view of the screen relative to the eyes, and so on, making it more convenient to use.

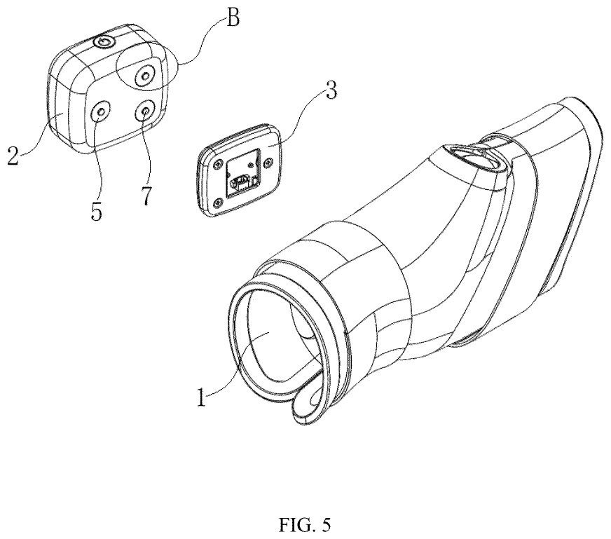

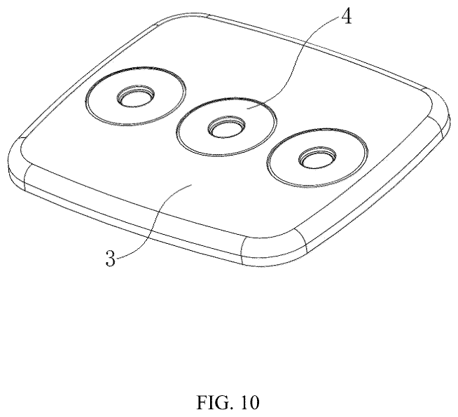

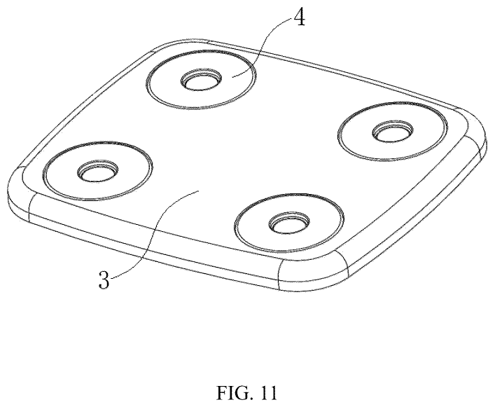

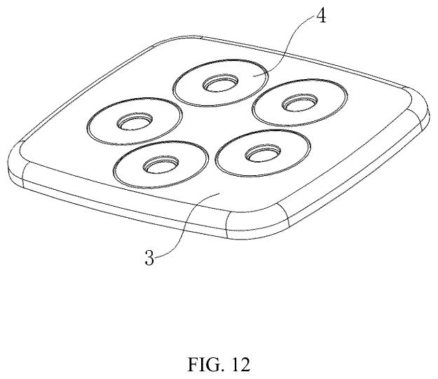

In some embodiments, the number of the first conductive components 4 and the number of the second conductive component 5 are the same, for example, both the number of the first conductive components 4 and the number of the second conductive component 5 are three (see FIG. 2 and FIG. 5), to form a triangular stability, or alternatively, the multiple first conductive components 4 and the multiple second conductive component 5 can be uniformly distributed, including linear array distribution, rectangular shape distribution, circular shape distribution, and other arrangement methods. Referring to FIG. 10, when multiple first conductive components 4 are distributed in a linear array, the control device 2 can be mounted to the base 3 in two opposite directions. Referring to FIG. 11, the multiple first conductive components 4 can be distributed in a rectangular shape. In other alternative embodiments, the multiple first conductive components 4 can be distributed in a square shape, and in the square shape, the control device 2 can be mounted to the base 3 in four equivalent directions. Referring to FIG. 12, when the multiple first conductive components 4 are distributed in a circular array, the number of installation positions of the control device 2 relative to the base 3 is the same as the number of first conductive components 4.

In other embodiments, both the number of the first conductive components 4 and the number of the second conductive component 5 can be only one, it can satisfy a detachable connection, for example, in the non-circular shape described above; or alternatively, the first conductive components 4 comprises multiple first conductive components 4, and the number of the second conductive component 5 is only one, and the second conductive component 5 is selectively connected to one of the multiple first conductive components 4.

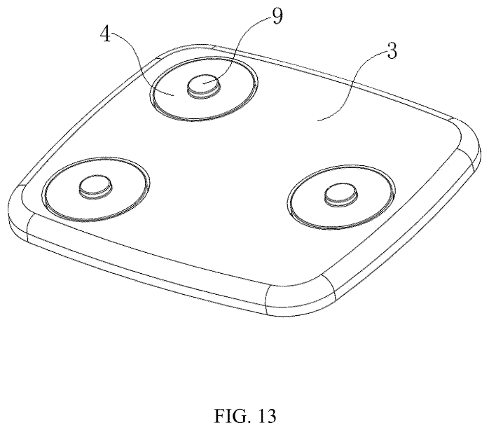

There are other ways of plugging and unplugging connections, for example, referring to FIG. 13, the middle of the first conductive component 4 is provided with a second protrusion 9, and the second conductive component 5 is embedded in the control device 2. Referring to FIG. 13, the middle of the second conductive component 5 is provided with a second groove 10 corresponding to the second protrusion 9. The first conductive component 4 and the second conductive component 5 can be plugged and unplugged through the cooperation of the second protrusion 9 and the second groove 10.

Through the design of the above structure, the plug-in connection between the first conductive component 4 and the second conductive component 5 can still be completed. At this time, since the second conductive component 5 is embedded in the control device 2 and there are no protruding parts on the surface of the control device 2, the flatness of the control device 2 can be maintained, making it more stable to place after disassembly.

Referring to FIG. 15, the energy storage component 21 is a lithium-ion battery, or a lithium-ion battery pack, the lithium-ion battery and the lithium-ion battery pack provides a voltage in a range of 2.4-4.2V.

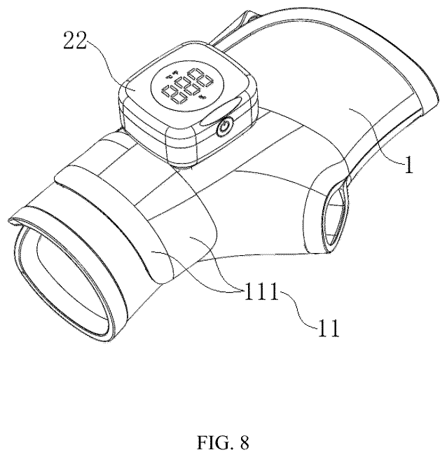

Referring to FIGS. 7-9, in order to better understand how the therapy component 11 is installed on the wrist guard body 1, this embodiment introduces one of the structures. Specifically, the wrist guard body is provided with a through hole 105, and the therapy component 11 includes a connecting part 111 and a therapy part 112. The size of the connecting part 111 is greater than that of the through hole 105, and the connecting part 111 is located on the outer surface of the wrist guard body 1 and corresponds to the through hole 105; the size of the therapy part 112 is less than or equal to the size of the through hole 105. The therapy part 112 is set on the connecting part 111 and located inside the through hole 105, used to fit the hand for therapy.

Based on the above scheme, since the size of the connecting part 111 is larger than that of the through hole 105, the size of the connecting part 111 is significantly larger, which is convenient for installation and being bound and fixed by the strap 103.

Furthermore, the therapy part 112 can be equipped with heating plates, red light plates, etc.

It should be noted that the terms used here are only intended to describe specific embodiments and are not intended to limit the exemplary embodiments according to the present application. As used herein, unless otherwise explicitly stated in the context, the singular form is also intended to include the plural form. Additionally, it should be understood that when the terms “including” and/or “including” are used in this specification, they indicate the presence of features, steps, operations, devices, components, and/or combinations thereof.

In addition, it should be noted that the use of words such as “first” and “second” to define components is only for the purpose of distinguishing the corresponding components. If not otherwise stated, the above words do not have a special meaning and therefore cannot be understood as limiting the scope of protection of this application.

Although embodiments of the present invention have been shown and described, it can be understood by those skilled in the art that multiple variations, modifications, substitutions, and variations can be made to these embodiments without departing from the principles and spirit of the present invention. The scope of the present invention is limited by the accompanying claims and their equivalents.

What is claimed is:

1. A physical therapy wrist guard, comprising:
a wrist guard body suitable for wrapping around a hand, and providing a therapy component for treating the hand, the wrist guard body comprising a base corresponding to the back of the hand, and at least one first conductive component arranged in the base, and being electrically connected to the therapy component; and
a control device comprising at least one second conductive component configured for being detachably connected to the at least one first conductive component, and an energy storage component being electrically connected to the at least one second conductive component, after the at least one second conductive component is connected to the at least one first conductive component, the energy storage component supplies energy to the therapy component.

2. The physical therapy wrist guard according to claim 1, wherein an extension direction of the at least one first conductive component is perpendicular to a surface where the base is located, and an extension direction of the at least one second conductive component is perpendicular to a surface of the control device facing the base, the at least one second conductive component is detachably connected to the at least one first conductive component by insertion and removal, to restrict the movement of the control device within the surface where the base is located.

3. The physical therapy wrist guard according to claim 1, wherein the at least one first conductive component and the at least one second conductive component are both made of magnetic material and are magnetically connected, to restrict the movement of the control device in the direction perpendicular to the surface of the base, so that after the at least one second conductive component is connected to the at least one first conductive component, the relative position between the control device and the base is fixed.

4. The physical therapy wrist guard according to claim 1, wherein the at least one first conductive component is embedded in the base, and a middle of the at least one first conductive component is provided with a first groove, a middle of the at least one second conductive component is provided with a first protrusion corresponding to the first groove, the at least one second conductive component and the at least one first conductive component can be plugged and connected through the cooperation of the first groove and the first protrusion.

5. The physical therapy wrist guard according to claim 4, wherein a side of the first groove away from the wrist guard body and a side of the first protrusion away from the control device both have a chamfer to facilitate the insertion of the first protrusion into the first groove.

6. The physical therapy wrist guard according to claim 4, wherein a cross-section of the first groove is circular, and the at least one first conductive component comprises two first conductive components.

7. The physical therapy wrist guard according to claim 4, wherein a cross-section of the first groove is non-circular.

8. The physical therapy wrist guard according to claim 1, wherein the at least one first conductive component comprises multiple first conductive components, so that the second conductive component can be connected to the first conductive components in different directions, thereby changing the installation direction of the control device.

9. The physical therapy wrist guard according to claim 1, wherein a middle of the at least one first conductive component is provided with a first groove, a middle of the second conductive component is provided with a first protrusion corresponding to the first groove, the at least one first conductive component and the second conductive component can be plugged and connected through the cooperation of the first groove and the first protrusion.

10. The physical therapy wrist guard according to claim 1, wherein the energy storage component is a lithium-ion battery or a lithium-ion battery pack, and the lithium-ion battery and the lithium-ion battery pack provides a voltage in a range of 2.4-4.2V.

11. The physical therapy wrist guard according to claim 3, wherein a magnetic force between the at least one first conductive component and the at least one second conductive component is greater than a gravity of the control device.

12. The physical therapy wrist guard according to claim 1, wherein the at least one first conductive component comprises multiple first conductive components, the at least one second conductive component comprises multiple second conductive components, the multiple first conductive components and the multiple second conductive components are uniformly distributed, including linear array distribution, square shape distribution, and circular shape distribution.

13. The physical therapy wrist guard according to claim 1, wherein the wrist guard body is provided with a through hole, and the therapy component is mounted in the through hole.

14. The physical therapy wrist guard according to claim 13, wherein the therapy component comprises a connecting part and a therapy part, a size of the connecting part is greater than that of the through hole, such that the connecting part is mounted on the wrist guard body, and a size of the therapy part is less than that of the through hole, such that the therapy part is located in the through hole to close to the hand.

15. A physical therapy wrist guard, comprising:
a wrist guard body suitable for wrapping around a hand, and providing a therapy component for treating the hand, the wrist guard body comprising a base corresponding to the back of the hand, and at least one first conductive component arranged in the base, and being electrically connected to the therapy component; and
a control device comprising at least one second conductive component configured for being detachably connected to the at least one first conductive component, and at least one operation button to operate the wrist guard body.
